(19) United States
(12) Patent Application Publication
(10) Pub. No.: US 2011/0076706 A1
(43) Pub. Date: Mar. 31, 2011

(54) METHODS AND KITS FOR THE RAPID DETECTION OF MICROORGANISMS

(75) Inventors: James E. Fleming, Spokane, WA (US); Claire Norton, Spokane, WA (US); Cheryl Fleming, Spokane, WA (US)

(73) Assignee: GENPRIME, INC., Spokane, WA (US)

(21) Appl. No.: 12/823,810

(22) Filed: Jun. 25, 2010

Related U.S. Application Data
(60) Provisional application No. 61/220,837, filed on Jun. 26, 2009.

(51) Int. Cl. C12Q 1/66; G01N 1/30; C12M 1/34

(52) U.S. Cl. 435/8; 435/40.5; 435/287.1

(57) ABSTRACT

The present invention provides analytical methods and related kits for detecting the presence of bacteria in a sample, determining the amount of bacteria in a sample, or determining the type of bacteria in a sample. In particular, the invention relates to devices and methods suitable for the rapid detection, quantification, or identification of bacteria in a liquid sample by measuring bacterial ATP activity.

### CROSS-REFERENCE TO RELATED APPLICATION

This application claims the benefit under 35 U.S.C. §119(e) of U.S. Provisional Patent Application No. 61/220,837 filed Jun. 26, 2009, where this provisional application is incorporated herein by reference in its entirety.

### BACKGROUND

1. Technical Field

The present invention relates to analytical methods for detecting the presence of bacteria in a sample, determining the amount of bacteria in a sample, and determining the type of bacteria in a sample. In particular, the invention relates to devices and methods suitable for the rapid detection of bacteria in a liquid sample.

2. Description of the Related Art

Bacterial contamination of water, beverages, food products, cosmetic and health care products, pharmaceutical products, and other products ingested or used by humans or other animals is a relatively common source of infection and associated disease and is, consequently, a large concern for industrial manufacturers. Bacterial contamination is of particular concern to industries that produce and sell food and beverages, blood-related products, and pharmaceutical preparations, since these are directly introduced into the human or animal body. While the sources of bacterial contamination vary between industries, all industries attempt to minimize contamination and detect any contamination prior to the sale or use of the contaminated product.

For example, bacterial contamination of platelets is a significant problem in the blood banking industry. Platelets are small anuclear blood cells derived from megakaryocytes in the bone marrow. They function in combination with fibrin and various cellular mechanisms to inhibit bleeding by forming haemostatic plugs in vessel walls (1). Patients with reduced platelet count caused by cancer chemotherapy, organ transplantation, various surgeries, and trauma often receive transfusions to prevent or stop bleeding (2). This is a critical treatment for many patients and over 9 million platelet concentrate equivalent units are transfused in the U.S. every year (3).

Prior to transfusion, blood banks and other centers routinely store platelets at ambient temperatures (20-25° C.) with gentle agitation under aerobic conditions (4). Storage under these conditions, which is required to maintain the efficacy of platelets, unfortunately provides an ideal growth environment for bacteria following an inadvertent contamination (5, 6). Although bacterial growth can occur in any blood product, the unique storage conditions of platelet suspensions make them the most susceptible to serious contamination (7). This is supported by clinical data that shows that platelets are the most contaminated transfused blood product with a rate of infectious risk occurring in approximately 1 in 2,000 to 1 in 3,000 individuals. These include transfusions from both whole-blood derived random donor platelets and apheresis-derived single donor platelets (7). Mortality rates have been estimated to range from 1 in 20,000 to 1 in 85,000, which is somewhere between 100 and 150 transfused individuals per year (7).

The clinical significance of this data stimulated the American Association of Blood Banks (AABB) to issue the following mandate to all of its member institutions. “The blood bank or transfusion service shall have method(s) to limit and detect bacterial contamination in all platelet components . . . . Standard 5.1.5.1 shall be implemented by Mar. 1, 2004.” According to this standard, all member blood banks must test every bag of platelets for bacterial contamination prior to transfusion. The pre-transfusion detection of bacteria undoubtedly represents an important step in reducing transfusion-related sepsis. Unfortunately, no current single detection technique is suitable for both types of platelets under all conditions (7).

A number of centers now carry out a variety of rapid methods of detection such as gram staining, platelet swirling, and the use of pH/Glucose dipsticks to estimate the bacterial load of platelets (7). All of these methods are low-sensitivity techniques, which may be inadequate to prevent sepsis-induced reactions following transfusion (7). Despite the widespread use of these methods, especially the pH/Glucose dipsticks, the clinical data suggest that they have not been very useful in detecting bacterial concentrations known to cause sepsis (7). Most likely, hospitals and blood centers implemented the pH/glucose test strip in order to maintain compliance with the AABB mandate; however, it appears that it may only provide a facility with a false sense of security.

Currently, there are only two systems routinely employed in U.S. blood banks which are FDA cleared for bacterial detection in platelets. These are the eBDS by Pall Corporation (East Hills, N.Y., USA) and the BacT/ALERT by bioMerieuex (Marcy l'Etoile, France). Both devices are culture-based technologies that have good sensitivity for detection of bacteria in platelets (8). The eBDS measures O₂ consumption, and the BacT/ALERT measures CO₂ production. The manufacturers of these devices estimate their sensitivity to be in the range of 1-10 CFU/mL. Unfortunately, neither of these devices can be rapidly used in a hospital transfusion center to assess bacterial load immediately prior to a transfusion. This is because these systems require that platelet products be held for 24 hours before sampling and another 24 hours of culturing before results are obtained (7).

Hemosystems (Marseilles, France) has developed a DNA-specific fluorescent labeling system for the detection of bacteria in platelets (8). The method concentrates bacteria on a membrane surface followed by quantification using solid-phase cytometry. This device is not currently marketed in the United States but has FDA clearance for sale in the United States. However, the method takes approximately 90 minutes for results, it is costly, and the test is difficult to perform, thus making it inadequate for routine screening in blood banks and hospitals.

Verax Biomedical (Worcester, Mass., USA) has developed a rapid lateral flow device for measuring bacteria in platelets called the PGD (Pangenera detection). It is based on the detection of conserved antigens in bacteria, specifically lipoteichoic acid on gram-positive bacteria and lipopolysaccharides on gram-negative bacteria. This test is cleared by the FDA for sale, but can only be used as an adjunct test in combination with the eBDS or BacT/Alert.

It is clear that there is an urgent requirement for a rapid bacterial test with adequate sensitivity to replace the insensitive, non-specific, and unregulated pH/Glucose tests that are currently employed to test whole blood platelets. The AABB has stated that they strongly encourage and support the development of such tests (9). This will permit their member centers to comply with the mandate using FDA regulated bacterial detection devices. Similarly, there is a need for a rapid bacterial test for routine testing of other products, including water, food, beverages, and pharmaceuticals.

The current invention solves this and other problems by providing a robust, rapid and sensitive test for bacterial contamination.

### BRIEF SUMMARY

The present invention provides methods and compositions for detecting the presence of bacteria in a sample, determining the amount of bacteria in a sample, and determining the type of bacteria present in a same.

In one embodiment, the present invention includes a method for detecting the presence of bacteria in a sample, comprising: determining a first amount of ATP activity present in a sample; subsequently contacting the sample with a lytic agent at a concentration and for a time sufficient to lyse bacteria in said sample; and determining a second amount of ATP activity present in the sample concurrent with or following contacting the sample with the lytic agent, thereby determining the presence of bacteria in the sample.

In another embodiment, the present invention includes a method for determining an amount of bacteria in a sample, comprising: determining a first amount of ATP activity present in a sample; subsequently contacting the sample with a lytic agent at a concentration and for a time sufficient to lyse bacteria in said sample following step (a); and determining a second amount of ATP activity present in the sample concurrent with or following contacting the sample with the lytic agent, thereby determining the amount of bacteria in the sample.

In particular embodiments, methods of the present invention may also include reducing the first amount of ATP activity. In one embodiment, the first amount of ATP activity is reduced before determining the first amount of ATP activity. In one embodiment, the first amount of ATP activity is reduced after determining the first amount of ATP activity and before contacting the sample with the lytic agent.

In particular embodiments, methods of the present invention may further comprise contacting the sample with a lytic agent at a concentration and for a time sufficient to lyse mammalian cells but not bacteria before determining the first amount of ATP activity.

In another embodiment, the present invention includes a method for determining the presence of bacteria in a sample, comprising: reducing ATP activity present in a sample; subsequently contacting the sample with a lytic agent at a concentration and for a time sufficient to lyse bacteria in said sample; and determining an amount of ATP activity present in the sample concurrent with or following contacting the same with the lytic agent, thereby determining the presence of bacteria in the sample.

In another embodiment, the present invention includes a method for determining an amount of bacteria in a sample, comprising: reducing ATP activity present in a sample; subsequently contacting the sample with a lytic agent at a concentration and for a time sufficient to lyse bacteria in said sample; and determining an amount of ATP activity present in the sample concurrent with or following contacting the sample with the lytic agent, thereby determining an amount of bacteria in the sample.

In particular embodiments, any of the methods of the present invention may further comprise a subsequent step of determining the presence of, the amount of, or the type of bacteria present in the sample.

In another embodiment, the present invention includes a method for identifying the type of bacteria in a sample, comprising: reducing ATP activity present in a sample; subsequently contacting the sample with a lytic agent at a concentration and for a time sufficient to lyse bacteria in said sample; and determining the rate of ATP activity present in the sample concurrent with or following contacting the sample with the lytic agent, thereby identifying the type of bacteria in the sample. In particular embodiments, the method further comprises a subsequent step of identifying the type of bacteria present in the sample.

In a related embodiment, the present invention includes a method for identifying the type of bacteria in a sample, comprising: determining a first amount of ATP activity in a sample; subsequently contacting the sample with a lytic agent at a concentration and for a time sufficient to lyse bacteria in said sample; and determining the rate of ATP activity present in a sample concurrent with or following contacting the sample with the lytic agent, thereby identifying the type of bacteria in the sample. In particular embodiments, the method further comprises a subsequent step of identifying the type of bacteria present in the sample.

In another embodiment, the present invention includes a method for detecting the presence of bacteria in a sample, comprising contacting a sample with a lytic agent at a concentration and for a time sufficient to lyse bacteria in said sample and concurrently determining an amount of ATP activity present in the sample.

In various embodiments of the present invention, the sample is a liquid sample. In certain embodiments, the liquid sample is a biological sample. In particular embodiments, the biological sample is blood, plasma, urine, platelets, bronchial lavage, saliva, wound fluid, tears, cerebrospinal fluid, amniotic fluid, or pleural fluid. In certain embodiments, the liquid sample is a food or beverage. In particular embodiments, the food or beverage is a dairy product. In one embodiment, the beverage is an alcoholic beverage. In certain embodiments, the alcoholic beverage is beer. In one embodiment, the liquid sample is fermenting corn mash. In another embodiment, the liquid sample is a cell culture.

In certain embodiments of the present invention, the amounts of ATP activity are determined using a luciferin/luciferase reagent.

In particular embodiments of the present invention, contacting the sample with a lytic agent to lyse bacteria and determining the second amount of ATP activity present in the sample are performed concurrently. In certain embodiments, these steps are performed concurrently using a luciferin/luciferase reagent containing a lytic reagent.

In certain embodiments of the present invention, the presence, amount, or type of bacteria is determined using time-resolved light detection to determine an ATP kinetic profile.

In one embodiment, the presence of bacteria is determined where the second amount of ATP activity is significantly greater than the first amount of ATP activity. In another embodiment, the amount of bacteria is determined by a method comprising subtracting the first amount of ATP activity from the second amount of ATP activity. In another embodiment, the amount of bacteria is determined by a method comprising dividing the second amount of ATP activity by the first amount of ATP activity.

In various embodiments of the present invention, the bacteria is Staphylococcus epidermis, Staphylococcus aureus, Bacillus cereus, Bacillus subtilis, Pseudomonas aeruginosa, Steptococcus viridans/mitis, Streptococcus pyogenes, Escherichia coli, Klebsiella oxytoca, Serratia marcescens, Enterobacter cloacae, Clostridium perfringens, Corynebacterium xerosis, Propionibacterium acnes, Salmonella spp., Lactobacillus rhamnosus, Lactobacillus fermentum, Lactobacillus paracasei, Lactobacillus brevis, Lactobacillus acidophilus, Lactobacillus plantarum, Lactobacillus crispatus, Lactobacillus delbrueckii, Pediococcus acidilactici, Pediococcus pentosaceus, Weisella confusa, Leuconostoc citreum, Leuconostoc mesenteroides, Leuconostoc lactis, Acetobacter spp., Gluconobacter oxydans, Serratia liquefaciens, klebsiella pheumoniae, Providencia stuartii, Providencia rettgeri, Providencia alcalifaciens, Proteus mirabilis, Proteus vulgaris, Morganella morganii, or Enterococcus faecalis.

In additional embodiments, the methods of the present invention further comprise providing a bacterial sample obtained from a liquid.

### BRIEF DESCRIPTION OF THE SEVERAL VIEWS OF THE DRAWINGS

FIG. 1 is a graph showing kinetic curves of ATP bioluminescence of different species of bacteria that have been spiked into whole blood-derived (WBD) platelet concentrates and a typical curve of non-contaminated WBD platelets (platelets only). Kinetic curves were created by the addition of a lytic reagent containing luciferin/luciferase that reacts with the exposed ATP. All samples were treated by a novel separation process of the present invention to remove most of the non-bacterial cells and extracellular ATP. On this graph, lytic reagents are shown as added at time zero. This graph demonstrates the different rates of ATP release from these different species of bacteria.

FIG. 2 is a graph showing the pattern of ATP bioluminescence in WBD platelets spiked with 1×10⁴ CFU/mL of Enterobacter cloacae and WBD platelets without bacterial contamination (platelets only). All samples were treated by a novel separation process of the present invention that removed most of the non-bacterial cells and extracellular ATP. The sample was then treated with a custom luciferin/luciferase reagent without lytic reagents to assess background ATP. At 31 seconds, BacTiter-Glo™ containing bacterial lytic reagents was injected into the sample. The peak shows the relative level of intracellular bacterial ATP released into the sample compared to the residual extracellular ATP.

FIG. 3 is graph showing the pattern of ATP bioluminescence in WBD platelets spiked with 5×10⁴ CFU/mL, 1×10⁴ CFU/ml or 0 CFU/mL of Pseudomonas aeruginosa. A novel separation process of the present invention that removes most of the non-bacterial cells and extracellular ATP was used on all samples. The sample was then treated with a luciferin/luciferase reagent without lytic reagents to assess background ATP. At 62 seconds, BacTiter-Glo™ containing bacterial lytic reagents was injected into the sample. The peaks correspond to the relative level of ATP generated by P. aeruginosa in the sample.

FIG. 4 is a graph showing the kinetic curves of ATP bioluminescence in a finished beer product spiked with Lactobacillus acidophilus or Lactobacillus fermentum at 1×10⁴ CFU/mL, and the beer product without bacteria. The sample was initially treated with a luciferin/luciferase reagent without lytic reagents to assess background ATP. At 30 seconds, BacTiter-Glo™ containing lytic reagents was injected into the sample. The peak after injection divided by the level prior to injection permits a calculation of the relative level of bacteria in the sample.

FIG. 5 is a graph showing the kinetic curves of Lactobacillus acidophilus spiked into a finished beer product at concentrations ranging from 0 to 2×10⁴ CFU/mL. The sample was initially treated with a luciferin/luciferase reagent without lytic reagents to assess background ATP. At 30 seconds, BacTiter-Glo™ containing lytic reagents was injected into the sample. The peak after injection divided by the level prior to injection permits a calculation of the relative level of bacteria in the sample.

FIG. 6 is a graph showing the kinetic curve of ATP bioluminescence in yeast-fermenting corn mash and yeast-fermenting corn mash spiked with Lactobacillus brevis at a concentration of 5×10⁵ CFU/mL. The sample was treated using a novel separation process of the present invention that removes the viable yeast and most of the extracellular ATP from the sample. The sample was treated with a luciferin/luciferase reagent without lytic reagents to assess background ATP. At 60 seconds, BacTiter-Glo™ containing lytic reagents was injected into the sample. The peak after injection divided by the level prior to injection permits a calculation of the relative amount of bacteria in the sample.

FIG. 7 is a graph showing the kinetic curves of ATP bioluminescence of Lactobacillus brevis at a concentration of 5×10⁷ CFU/mL in GenPrime custom buffer using two different lysis methods. Both samples were treated with a luciferin/luciferase reagent without lytic reagents to assess background ATP. At 100 seconds, BacTiter-Glo™ containing lytic reagents was injected into the first sample labeled Detergent-lysis. At the same time point, a constant voltage with a field strength of 300 volts per millimeter was applied for 500 milliseconds to the second sample labeled Electro-lysis.

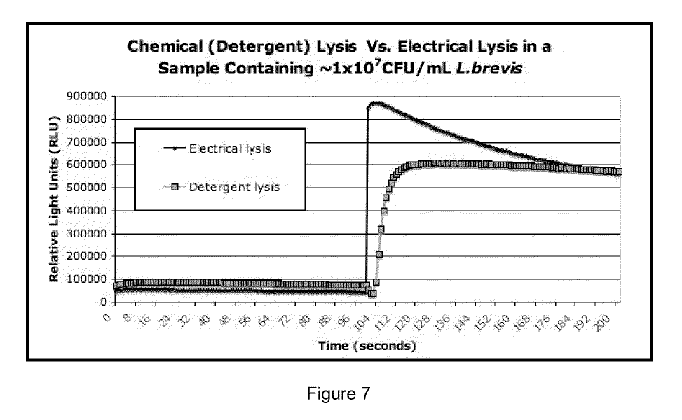

### DETAILED DESCRIPTION

The present invention provides methods and reagents useful in rapidly detecting bacterial contamination using ATP luminescence, as well as related methods for rapidly determining the amount of bacteria in a sample and the identity of bacteria in a sample. These methods are advantageous over the currently used methods, in part because they may be performed rapidly using only a small sample, and they do not require the culturing of bacteria. In particular embodiments, the methods and reagents of the present invention distinguish bacterial ATP from non-bacterial ATP, which is particularly advantageous for the testing of biological samples that contain eukaryotic, e.g., mammalian, cells.

The present invention may be used to detect the presence of bacteria in a wide variety of different samples. Therefore, the present invention may be used in a variety of clinical, manufacturing, and household settings. For example, the methods and kits of the present invention may be used by blood banks, hospitals, and transfusion centers prior to transfusion, to determine whether concentrated platelet units are contaminated with bacteria. It may also be employed in other clinical settings for determining bacterial contamination of various biological fluids, such as urine, saliva, and blood. In addition, the present invention may be used by food and beverage manufacturers, pharmaceutical manufacturers, healthcare product manufacturers, and others to test their products to ensure that they are not contaminated with bacteria. Furthermore, the present invention may be used in the household setting to test for bacterial contamination.

The present invention may be used to detect the presence of bacteria or other microorganisms, e.g., yeast, in a variety of samples. While the invention is typically used to detect bacteria in liquid samples, it may also be used to detect bacteria in solid samples that are suspended or dissolved in a liquid. Alternatively, a solid sample may be soaked in a liquid to release bacteria from the sample into the liquid, and the liquid may then be tested for the presence of bacteria. In addition, a non-liquid sample, such as a solid, e.g., a surface, may be swabbed and the swab then immersed in a liquid to release any bacteria into the liquid. In certain embodiments, a liquid sample may be diluted before being tested, e.g., in water or a buffer such as phosphate buffered saline (PBS).

The invention has wide applications, since it is able to rapidly detect bacteria in any sample, including, but not limited to, biological samples, food and beverages, and cell cultures. In certain embodiments, the invention may be used to detect bacteria associated with bacterial contamination of beverages and foods and/or infection of humans or other animals. In particular embodiments, the methods of the present invention may be used to detect the presence of bacteria in a sample in less than one minute, less than two minutes, less than five minutes, less than ten minutes, less than twenty minutes, less than thirty minutes, less than one hour, less than two hours, or less than four hours. In one particular embodiment, the ATP assay of the present invention is performed in about two to about three minutes. In another embodiment, bacteria may be detected in beer in about five minutes. In another embodiment, bacteria may be detected in a platelet preparation in about thirty minutes, including the time for sample preparation.

Examples of biological samples that may be tested using the present invention include, but are not limited to, urine, cerebrospinal fluid, saliva, amniotic fluid, vaginal fluid, semen, platelets, blood products, myocardial fluid, stomach acid/secretions, bronchial lavage, pleural fluid, lipoaspirates, stem cell samples, fecal matter, wound fluid, and tears. In various embodiments, blood products include blood, whole blood, plasma, platelets, red blood cells, or leukocytes, including concentrates suitable for transfusion.

Examples of food and beverages that may be tested using the present invention include dairy product, alcoholic beverages, and other products. Particular examples include, but are not limited to, juices, milk and milk products, breast milk, soy milk, beer, wine, canned foods, processed foods, liquid foods, well water, yogurt, liquified meat products, baby food, formula, and apple or other cider. A variety of food products may be tested, although depending upon the nature and consistency of the food product, it may need to be further processed prior to test, for example, by diluting or dissolving the food in a liquid. In one embodiment, the sample is fermenting corn mash.

Products and other samples may be tested at any stage in their production. For example, milk may be tested immediately after or shortly after being obtained from the animal or following further processing, such as pasteurization. Wine, for example, may be tested while it is still grape juice, at different stages of fermentation, or following sedimentation or filtration.

Water samples that may be tested include, but are not limited to, drinking or potable water. Water from any source may be tested according to the invention, including, e.g., water from swimming pools, heating and cooling systems, and natural or outdoor waters, e.g., lakes and rivers. Other water samples include, e.g., fluid obtained from dental water unit lines, RV water tanks, airplane water tanks, boat (e.g., cruise ship) water tanks, refrigerator water lines, fish tanks, and animal water.

Additional samples that may be tested according to the present invention include, but are not limited to, cosmetics, cell cultures (e.g., media from a mammalian cell culture), laboratory reagents, pharmaceutical products, health care products, petroleum products, and waste water.

The present invention may be used to detect the presence of any bacteria, including aerobic and anaerobic bacteria, and gram positive and gram negative bacteria.

In certain embodiments, the present invention is used to detect the presence of aerobic or anaerobic bacteria. Examples of aerobic bacteria associated with blood infections and related disease include, amongst others, Neisseria, Pseudomonas aeruginosa, Staphylococcus aureus, Klebsiella pneumoniae, Serratia marcescens, and Staphylococcus epidermidis. Other organisms that may be detected include, e.g., Salmonella sp., Escherichia coli, Pseudomonas aeruginosa, and Bacillus cereus. Examples of anaerobic bacteria that may cause infections of the blood include, but are not limited to, Bacteroides, E. coli, Klebsiella, and Clostridium. It is further understood that detection can be accomplished independent of the genus or species. Thus, any microorganism that comprises ATP may be detected according to the invention.

In other embodiments, the invention is used to detect the presence of gram positive or gram negative bacteria, including pathogenic bacteria, e.g., in blood products, beverages or food products. In some cases, milk can contain pathogenic bacteria, such as E. coli, salmonella, and listeria. For example, research conducted by the British government recently found contamination with a form of tuberculosis bacterium in about 10% of samples of milk. The bacterium, mycobacterium avium paratuberculosis (MAP), has been linked to Crohn's disease in humans.

Examples of other bacteria that may be detected according to the present invention include, but are not limited to, the gram-negative strains: Spirochaeta sp, Cristispira sp, Treponema sp, Borrelia sp, Leptospira sp, Campylobacter sp, Spirillium sp, Spirosoma sp, Pseudomonas sp, Xanthomonas sp, Phisobium sp, Methylococcus sp, Halobacterium sp, Acetobacter sp, Legionella sp, Neisseria sp, Moraxella sp, Flavobacterium sp, Brucella sp, Bordetrella sp, Francisella sp, Escherichia sp, Shigella sp, Salmonells sp, Citrobacter sp, Klebsiella sp, Enterobacter sp, Erwinia sp, Serratia sp, Hafnia sp, Edwardsiella sp, Proteus sp, Providencia sp, Morganella sp, Yersina sp, Vibrio sp, Pasterurella sp, Haemophilus sp, Desulfuromanas sp, Desulfovibrio sp, Desulfomanonas sp, Desulfococcus sp, Desulfobacter sp, Desulfobulbus sp, Desulfosarcina sp, Veillonella sp, Rickettsia sp, Rochalimeae sp, Coxiella sp, Ehrlichia sp, Cowdria sp, Wolbachia sp, Rickettsiella sp, Chlamydia sp, Mycoplasma sp, Ureaplasma sp, and Spiroplasma sp.

Examples of gram-positive bacteria that may be detected according to the present invention include, but are not limited to: Micrococcus sp, Stomatococcus sp, Planococcus sp, Staphlycoccus sp, Deinococcus sp, Streptococcus sp, Sarcina sp, Pediococcus sp, Bacillus sp, Sporolactobacillus sp, Clostridium sp, Desulfotomaculum, Sporosarcina sp, Gardnerella sp, Streptobacillus sp, Lactobacillus sp, Listeria sp, Erysipelothrix sp, Corynebacterium sp, Mycobacterium sp, Nocardia sp, Haemophillus sp, and Heliobacter sp.

Particular species of bacteria that may be detected, quantified, or identified according to various embodiments of the present invention include, but are not limited to, Staphylococcus epidermis, Staphylococcus aureus, Bacillus cereus, Bacillus subtilis, Pseudomonas aeruginosa, Steptococcus viridans/mitis, Streptococcus pyogenes, Escherichia coli, Klebsiella oxytoca, Serratia marcescens, Enterobacter cloacae, Clostridium perfringens, Corynebacterium xerosis, Propionibacterium acnes, Salmonella spp., Lactobacillus rhamnosus, Lactobacillus fermentum, Lactobacillus paracasei, Lactobacillus brevis, Lactobacillus acidophilus, Lactobacillus plantarum, Lactobacillus crispatus, Lactobacillus delbrueckii, Pediococcus acidilactici, Pediococcus pentosaceus, Weisella confusa, Leuconostoc citreum, Leuconostoc mesenteroides, Leuconostoc lactis, Acetobacter spp., Gluconobacter oxydans, Serratia liquefaciens, klebsiella pheumoniae, Providencia stuartii, Providencia rettgeri, Providencia alcalifaciens, Proteus mirabilis, Proteus vulgaris, Morganella morganii, and Enterococcus faecalis.

The methods of the present invention provide for the rapid detection of a microorganism, e.g., bacteria, in a sample by detecting, determining, or measuring the microorganism's ATP activity. In particular embodiments, ATP activity is determined using an ATP-based bioluminescent assay. According to this assay, microorganisms, e.g., bacteria, are lysed and released ATP activity is determined. This determined ATP activity correlates with the number of microorganisms, e.g., bacteria, present in the sample, therefore allowing the detection of microorganism in a sample, as well as determining the amount of microorganism present in a sample. Any method of determining or measuring ATP activity may be employed according to the present invention. Such methods are known and available in the art, and include, e.g., bioluminescence-based assays. For example, ATP activity may be measured by measuring the presence or amount of any end product produced by a reaction that requires ATP as an energy source and can be performed in vitro. In one embodiment, high pressure liquid chromatography (HPLC) is used to measure ATP.

In particular embodiments of an ATP-based bioluminescent assay employed by the present invention, bacteria are contacted with a reagent that comprises a lytic agent capable of lysing bacteria, as well as luciferin, luciferase, and Mg²⁺. ATP released from the lysed bacteria allow for the mono-oxygenation of luciferin by luciferase in the presence of a divalent cation (such as Mg²⁺ or Ca²⁺) and molecular oxygen, according to the reaction shown below:

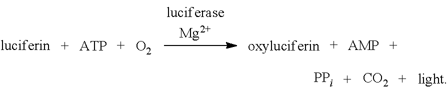

This reaction is extremely slow in the absence of ATP, so light is only generated in the presence of ATP. In particular embodiments, the luciferin is beetle luciferin; in particular embodiments, the luciferase is recombinant firefly luciferase. In particular embodiments, the luciferase is a thermostable luciferase such as the luciferase from the firefly, Photuris pennsylvanica. Various luciferase assays and suitable luminogenic reagents and conditions are known in the art, which may be readily used or adapted for us according to the present invention. See, e.g., Fan F, Wood K V (February 2007), “Bioluminescent assays for high-throughput screening”. Assay Drug Dev Technol 5 (1): 127-36. doi:10.1089/adt.2006.053. PMID 17355205; Meisenheimer P L, O'Brien M A, Cali J J (September 2008), “Luminogenic enzyme substrates: The basis for a new paradigm in assay design,” Promega Notes 100: 22-26 at http://www.promega.com/pnotes/100/16620_{—}22/16620_{—}22.pdf; Fan, et al., Microbial ATP Extraction and Detection System, U.S. Pat. No. 7,422,868, Sep. 9, 2008; and Wood et al., Kits for Detection of ATP, U.S. Pat. No. 7,452,663, Nov. 18, 2008.

In one embodiment, the reagent comprising the lytic agent capable of lysing bacteria and the reagents utilized in the luciferase assay is the BacTiter-Glo™ Reagent (Promega Corp., Madison, Wis., USA). Other reagents that may be used include, e.g., ATP Bioluminescent Assay Kit (Sigma-Aldrich, St. Louis, Mo. USA), ATP Lite, Perkin-Elmer Life and Analytical Sciences, Inc. (Waltham, Mass., USA), ATP Luminescence Assay Kit A22066, Invitrogen Corporation (Carlsbad, Calif., USA), ATP-Glo™ Bioluminometric Cell Viability Assay Kit 30020-1, Biotium, Inc. (Hayward, Calif. USA), ATP Bioluminescence Assay Kit HSII, Roche (USA), and Bioluminescent Cell Viability Kit 1, PKCA577-K254, PromoKine (Heidelberg, Germany).

The light emitted during the luciferase assay may be detected and quantified by any suitable means, including, e.g., a luminometer. The resulting amount of emitted light may be used to determine the number of bacteria present in the sample tested. Emitted light may be measured at a single time point or multiple times over a period of time, e.g., 5 seconds, 10 seconds, 15 seconds, 30 seconds, 45 second, 1 minute, 2 minutes, 3 minutes, 4 minutes, 5 minutes, 10 minutes, 15 minutes or 30 minutes, or longer. The amount of emitted light (e.g., at a particular time point, at its highest level, or total) may be compared to reference standards correlating to various concentrations or amounts of bacteria, in order to determine the number or concentration of bacteria present in the sample tested. Reference standards may be prepared in advance or determined at the same time that the assay is being performed on a test sample. Assays may be performed in various formats, including, e.g., in single microfuge tubes or in multiwall-plates, which may be used for high throughput testing of multiple samples. The pattern of emitted light measured over multiple time points, e.g., over a time period of 30 or 60 seconds, may be used to identify the type of bacteria present in the sample by comparing the pattern of emitted light to reference patterns produced by various different bacteria. It has been demonstrated that different species of bacteria produce different patterns of emitted light over time. This may be due to variations in the time it takes for different species of bacteria to lyse.

In certain embodiments of methods of the present invention, background levels of ATP present in the sample being tested are determined before lysing the bacteria and determining the ATP activity associated with the bacteria itself. Background levels of ATP activity may be readily determined using a luciferase assay, such as those described above or known in the art. In one embodiment, the amount of background ATP activity determined is subtracted from the amount of total ATP activity determined following lysis of the bacteria, in order to determine the amount or pattern of ATP activity attributable to the bacteria.

Certain samples tested according to the present invention may include other non-bacterial cells that have ATP activity, such as eukaryotic cells that may be present in biological samples, which contribute to background ATP activity. Accordingly, in certain embodiments of the present invention, background ATP activity associated with non-bacterial cells present in the sample being tested is determined before lysing the bacteria and determining the ATP activity associated with the bacteria itself. In one embodiment, the background ATP activity associated with non-bacterial, e.g., eukaryotic or mammalian cells, present in the sample is determined by selectively lysing the non-bacterial cells under conditions that do not lyse bacteria present in the sample.

Reagents and conditions suitable for selectively lysing non-bacterial, e.g., eukaryotic or mammalian cells, as opposed to bacteria are known in the art and include, e.g., 0.16% to 5.0% Triton X-100. In one embodiment, 0.16% Triton X-100 is used to selective lyse non-bacterial, e.g., eukaryotic or mammalian, cells. In particular embodiments, reagents and methods to selectively lyse eukaryotic cells include, but are not limited to, SDS, NP-40, boiling, sonication, freeze-thaw, RIPA buffer (Thermo Scientific), and B-PER (Thermo Scientific).

In particular embodiment, reagents and methods to lyse bacterial cells include, but are not limited to, TCA (Trichloroacetic acid), PCA (perchloric acid), CTAB (Cetyl trimethylammonium bromide), DTAB (dodecyl trimethyl ammoniumbromide), ultrasonication, other nonionic, anionic and cationic detergents, lysozyme, and other bacterial specific lytic enzymes such as lysostaphin. In certain embodiments, reagents and methods to lyse bacteria according to the present invention include those described in U.S. Pat. No. 7,422,868. This patent describes ATP extracting agents that may be used to lyse bacteria according to the present invention including, but not limited to, CTAB, a quaternary ammonium salt. In preferred embodiments, CTAB is present in the reagent composition at a concentration between about 0.04%-0.15% (w/v). In another embodiment, the ATP extracting agents may include CHEX and an ethoxylated alkylphenol, such as Triton X-100. In preferred embodiments, CHEX is preferably between about 0.04%-0.16% (w/v) and the ethoxylated alkylphenol is present between about 0.25%-1.0% (w/v). In a particularly preferred embodiment, the reagent composition may include more than one ATP extracting agent. One preferred embodiment includes CHEX, (between about 0.04%-0.16% (w/v)); an ethoxylated alkylphenol, such as Triton X-100 (between about 0.25%-1.0% (w/v)); and a quaternary ammonium salt, such as CTAB, (between about 0.02%-0.08% (w/v)). In particular embodiments, the bacterial lysis agent does not substantially interfere with the luciferin/luciferase reaction.

In particular embodiments, bacteriophages such as T1-T7, which specifically infect and lyse E. coli, may be employed to detect this bacterial species in suspension. Other bacteriophages may be employed to detect other species, and certain viruses could be used to detect specific organisms in a mixture. For example, lytic enzymes from bacteriophages such as C1 phage lysin that infect group C Streptococci may be employed to lyse Streptococci bacteria, releasing their intracellular ATP. Other viral lysins could be used such as those isolated from Bacillus phages.

In particular embodiments, electrical methods are used to lyse bacteria and/or induce pore formation in the membrane, thereby releasing ATP. This may be achieved by placing electrodes of suitable material such as platinum in micro-centrifuge tubes containing bacteria suspended in a solution such as GenPrime custom buffer. An electric pulse of from 100 to 10,000 volts per centimeter is applied for a time sufficient to release ATP. Time of the electric pulse can be 100 nanoseconds, 1 millisecond, 10 milliseconds, 100 milliseconds, 500 milliseconds, up to five minutes. Increased voltages, longer time pulses or multiple pulses may be used to selectively lyse and/or induce membrane pore formation for in certain bacteria.

Following selective lysis of non-bacterial, e.g., eukaryotic, cells present in the sample, the background ATP activity associated with these cells may be readily determined using a luciferase assay, such as those described above or known in the art. In one embodiment, the amount of background ATP activity associated with non-bacterial cells is subtracted from the amount of total ATP activity determined following lysis of the bacteria, in order to determine the amount or pattern of ATP activity attributable to the bacteria. In addition, in certain embodiments, both background ATP activity determined in the absence of any cell lysis and background ATP activity associated with non-bacterial cells may be determined in succession, or the combined amount of background ATP activity in the absence of any cell lysis and background ATP activity associated with non-bacterial cells may be determined at the same time. These background levels of ATP activity may then be subtracted from the total amount of ATP activity determined following lysis of bacteria to determine the amount of ATP activity attributable to the bacteria.

As described above, in certain embodiments, methods of the present invention include measuring or analyzing both background ATP activity prior to lysis of bacteria and ATP activity after or during lysis of bacteria, in order to determine bacterial ATP activity. As used herein, “background ATP activity” refers to ATP activity present in a sample prior to lysis of the bacteria. For example, background ATP activity may be associated with eukaryotic or mammalian cells present in the sample. In other embodiments, methods of the present invention comprise only measuring or analyzing ATP activity after or during lysis of bacteria. In either of these embodiments, background ATP activity may be reduced or removed prior to measuring bacterial ATP activity by contacting the sample with an agent that degrades or reduces ATP activity, such as, e.g., an apyrase or ATP diphosphohydrolase for a time and under conditions suitable to reduce ATP activity. Such reagents are known and available in the art, e.g., Sigma A6535 (Sigma-Aldrich, St. Louis, Mo., USA). Background ATP activity may be reduced or removed prior to measuring background ATP activity or after measuring any background ATP activity. After treatment with an agent that degrades or reduces background activity, the bacteria may optionally be washed to remove the agent before lysis of the bacteria, in order to prevent degradation or reduction of bacterial ATP activity.

According to methods of the present invention, a sample being tested may be a liquid sample. In particular embodiments, any bacteria present in the sample are collected and optionally washed prior to testing. The bacteria may also optionally be concentrated or diluted prior to testing. Bacteria may be collected from a liquid sample by any means available. For example, bacteria may be collected from a liquid sample by centrifuging the sample to collect a pellet of bacteria. This pellet may optionally be washed in water or an appropriate buffer that does not lyse the bacteria, and resuspended in solution prior to testing. Bacteria may also be separated from other components, e.g., solids or eukaryotic cells, of a liquid sample before testing, e.g., by filtering a liquid sample to collect other components while allowing the bacteria to pass through the filter, e.g., using a Swiss Gold coffee (SGC) filter and/or a Microcon® (5 um pore size) centrifugal filter tube, after which the bacteria may be collected, e.g., by centrifugation, and optionally washed.

In one embodiment, the present invention includes a method for detecting the presence of bacteria in a sample, comprising: determining a first amount of ATP activity present in a sample; subsequently contacting the sample with a lytic agent at a concentration and for a time sufficient to lyse any bacteria present in the sample; and then determining a second amount of ATP activity present in the sample concurrent with or following contacting the sample with the lytic agent. In particular embodiments, the time sufficient to lyse any bacteria in the sample is less than 1 second, about 1 second, about 2 seconds, about 5 seconds, about 10 seconds, about 15 seconds, about 20 seconds, about 30 seconds, about 45 seconds, about one minute, about two minutes, between two and five minutes, between 10 seconds and one minute, between one and ten minutes, about 10 minutes, about 20 minutes, about 30 minutes, about 40 minutes, between less than a second to 5 seconds, between 5 seconds and 5 minutes, between 5 minutes and 15 minutes, or between 15 minutes and 30 minutes. The presence of bacteria is indicated when the second amount of ATP activity is greater than the first amount of ATP activity, if the first amount of ATP activity is not reduced, e.g., by treatment with apyrase, before determining the second amount of ATP activity. If the first amount of ATP activity is reduced before determining the second amount of ATP activity, then the presence of bacteria is indicated when the second ATP activity is greater than zero, or greater than the ATP activity produced by a negative control that lacks any bacteria or other source of ATP. As discussed above, determining an amount of ATP activity present in a sample may include either determining an amount at a single time point or at multiple time points over a period of time, thereby producing a profile of ATP activity.

In another embodiment, the present invention includes a method for determining an amount of bacteria in a sample, comprising: determining a first amount of ATP activity present in a sample; subsequently contacting the sample with a lytic agent at a concentration and for a time sufficient to lyse any bacteria in said sample following step (a); and then determining a second amount of ATP activity present in the sample concurrent with or following contacting the sample with the lytic agent. The amount of bacteria may be determined by subtracting the first amount of ATP activity from the second amount of ATP activity, if the first amount of ATP activity is not reduced, e.g., by treatment with apyrase, before determining the second amount of ATP activity, and then comparing this amount to a control reference. If the first amount of ATP activity is reduced before determining the second amount of ATP activity, then the amount of bacteria is determined by comparing the second amount of ATP activity to appropriate control references.

In another embodiment, the present invention includes a method for determining the presence of bacteria in a sample, comprising: reducing ATP activity present in a sample; subsequently contacting the sample with a lytic agent at a concentration and for a time sufficient to lyse bacteria in said sample; and determining an amount of ATP activity present in the sample concurrent with or following contacting the same with the lytic agent. The presence of bacteria in the sample is indicated when the amount of ATP activity is greater than zero, or greater than the ATP activity produced by a negative control that lacks any bacteria or other source of ATP.

In another embodiment, the present invention includes a method for determining an amount of bacteria in a sample, comprising: reducing ATP activity present in a sample; subsequently contacting the sample with a lytic agent at a concentration and for a time sufficient to lyse bacteria in said sample; and determining an amount of ATP activity present in the sample concurrent with or following contacting the sample with the lytic agent. The amount of bacteria is determined by comparing the second amount of ATP activity to appropriate control references.

In a further embodiment, the present invention includes a method for detecting the presence of bacteria in a sample, comprising: contacting a sample, e.g., a biological sample, with an agent that preferentially lyses non-bacterial cells, e.g., eukaryotic cells; subsequently determining a first amount of ATP activity present in a sample; then subsequently contacting the sample with a lytic agent at a concentration and for a time sufficient to lyse any bacteria present in the sample; and then determining a second amount of ATP activity present in the sample concurrent with or following contacting the sample with the lytic agent. The presence of bacteria is indicated when the second amount of ATP activity is greater than the first amount of ATP activity, if the first amount of ATP activity is not reduced, e.g., by treatment with apyrase, before determining the second amount of ATP activity. If the first amount of ATP activity is reduced before determining the second amount of ATP activity, then the presence of bacteria is indicated when the second ATP activity is greater than zero, or greater than the ATP activity produced by a negative control that lacks any bacteria or other source of ATP.

In another embodiment, the present invention includes a method for determining an amount of bacteria in a sample, comprising: lysing eukaryotic cells present in the sample by contacting the sample, e.g., a biological sample, with an agent that preferentially lyses non-bacterial cells, e.g., eukaryotic cells; subsequently determining a first amount of ATP activity present in a sample; subsequently contacting the sample with a lytic agent at a concentration and for a time sufficient to lyse any bacteria in said sample following step (a); and then determining a second amount of ATP activity present in the sample concurrent with or following contacting the sample with the lytic agent. The amount of bacteria may be determined by subtracting the first amount of ATP activity from the second amount of ATP activity, if the first amount of ATP activity is not reduced, e.g., by treatment with apyrase, before determining the second amount of ATP activity, and then comparing this amount to a control reference. If the first amount of ATP activity is reduced before determining the second amount of ATP activity, then the amount of bacteria is determined by comparing the second amount of ATP activity to appropriate control references.

In another embodiment, the present invention includes a method for determining the presence of bacteria in a sample, comprising: lysing eukaryotic cells present in the sample by contacting the sample, e.g., a biological sample, with an agent that preferentially lyses non-bacterial cells, e.g., eukaryotic cells; subsequently reducing ATP activity present in a sample; subsequently contacting the sample with a lytic agent at a concentration and for a time sufficient to lyse bacteria in said sample; and determining an amount of ATP activity present in the sample concurrent with or following contacting the same with the lytic agent. The presence of bacteria in the sample is indicated when the amount of ATP activity is greater than zero, or greater than the ATP activity produced by a negative control that lacks any bacteria or other source of ATP.

In another embodiment, the present invention includes a method for determining an amount of bacteria in a sample, comprising: lysing eukaryotic cells present in the sample by contacting the sample, e.g., a biological sample, with an agent that preferentially lyses non-bacterial cells, e.g., eukaryotic cells; subsequently reducing ATP activity present in a sample; subsequently contacting the sample with a lytic agent at a concentration and for a time sufficient to lyse bacteria in said sample; and determining an amount of ATP activity present in the sample concurrent with or following contacting the sample with the lytic agent. The amount of bacteria is determined by comparing the second amount of ATP activity to appropriate control references.

In a further embodiment, the present invention includes a method for detecting the presence of bacteria in a sample, comprising: contacting a sample, e.g., a biological sample, with an agent that preferentially lyses non-bacterial cells, e.g., eukaryotic cells; subsequently reducing ATP activity present in a sample; subsequently determining a first amount of ATP activity present in a sample; then subsequently contacting the sample with a lytic agent at a concentration and for a time sufficient to lyse any bacteria present in the sample; and then determining a second amount of ATP activity present in the sample concurrent with or following contacting the sample with the lytic agent. The presence of bacteria is indicated when the second ATP activity is greater than zero, or greater than the ATP activity produced by a negative control that lacks any bacteria or other source of ATP.

In another embodiment, the present invention includes a method for determining an amount of bacteria in a sample, comprising: lysing eukaryotic cells present in the sample by contacting the sample, e.g., a biological sample, with an agent that preferentially lyses non-bacterial cells, e.g., eukaryotic cells; subsequently reducing ATP activity present in a sample; subsequently determining a first amount of ATP activity present in a sample; subsequently contacting the sample with a lytic agent at a concentration and for a time sufficient to lyse any bacteria in said sample following step (a); and then determining a second amount of ATP activity present in the sample concurrent with or following contacting the sample with the lytic agent. The amount of bacteria is determined by comparing the second amount of ATP activity to appropriate control references.

In a further embodiment, the present invention includes a method for detecting the presence of bacteria in a sample, comprising: contacting a sample, e.g., a biological sample, with an agent that preferentially lyses non-bacterial cells, e.g., eukaryotic cells; subsequently determining a first amount of ATP activity present in a sample; subsequently reducing ATP activity present in a sample; then subsequently contacting the sample with a lytic agent at a concentration and for a time sufficient to lyse any bacteria present in the sample; and then determining a second amount of ATP activity present in the sample concurrent with or following contacting the sample with the lytic agent. The presence of bacteria is indicated when the second ATP activity is greater than zero, or greater than the ATP activity produced by a negative control that lacks any bacteria or other source of ATP.

In another embodiment, the present invention includes a method for determining an amount of bacteria in a sample, comprising: lysing eukaryotic cells present in the sample by contacting the sample, e.g., a biological sample, with an agent that preferentially lyses non-bacterial cells, e.g., eukaryotic cells; subsequently determining a first amount of ATP activity present in a sample; subsequently reducing ATP activity present in a sample; subsequently contacting the sample with a lytic agent at a concentration and for a time sufficient to lyse any bacteria in said sample following step (a); and then determining a second amount of ATP activity present in the sample concurrent with or following contacting the sample with the lytic agent. The amount of bacteria is determined by comparing the second amount of ATP activity to appropriate control references.

The methods described herein may be readily adapted to determine the identity of bacteria present in a sample by comparing the profile of bacterial ATP activity generated by the sample to the profiles generated by one or more different species of bacteria, to identify the bacterial species that generates the same profile produced by the bacteria present in the sample. In particular embodiments, ATP activity is reduced, e.g., using apyrase, before lysing the bacteria, in order to remove any background ATP activity. In particular embodiments, eukaryotic cells present in the sample are lysed before reducing the ATP activity, by contacting the sample with an agent that preferentially lyses non-bacterial cells, e.g., eukaryotic cells, in order to remove any background ATP activity associated with eukaryotic cells present in the sample. In particular embodiments, the lysed eukaryotic cells and release ATP activity may be removed by washing the remaining bacteria, e.g., by pelleting and rinsing, and/or the ATP activity associated with the eukaryotic cells may be reduced by treatment with an agent such as apyrase. Profiles of ATP activity for various bacterial species may be readily generated by measuring the ATP activity over a period of time for various concentrations or amounts of known bacterial species, e.g., using a luciferase assay described herein.

In one particular embodiment, the present invention includes a method for the detection of bacteria in a biological sample, e.g., a platelet concentrate, where eukaryotic cells are first removed from the sample by a lysis step that preferentially lyses non-bacterial cells or eukaryotic cells. Detection is accomplished using a two-step ATP luminescence reaction, essentially as described above, where the first step consists of determining background ATP levels without the use of a bacterial lysis reagent, and the second step consists of determining ATP levels while incorporating a bacterial lytic step that releases intracellular ATP. Bacterial ATP may be calculated by subtracting total extracellular ATP determined during the first step from total ATP determined during the second step. The first step may include an ATP degrading enzyme such as apyrase to further reduce background ATP. In particular embodiments, bacteria can be identified by their unique ATP kinetic signal using time resolved light detection.

One example of how this particular embodiment of the present invention may be carried out is provided. A 1 mL platelet sample is collected, and the platelets are lysed with the addition of a lytic reagent (Triton X-100) at a concentration that does not affect bacteria. The sample is centrifuged at 7,500×g for 3 minutes to pellet any bacteria present in the sample. The supernatant is removed and the remaining pellet is incubated with an ATPase for 10 minutes at room temperature in order to degrade most of the extracellular ATP; apyrase may be used at a concentration of 495 units/mL. The apyrase activity is stopped by the addition of betaine, HCl, or heat, or the sample is centrifuged again at 7,500×g to pellet the bacteria and the resulting supernatant removed.

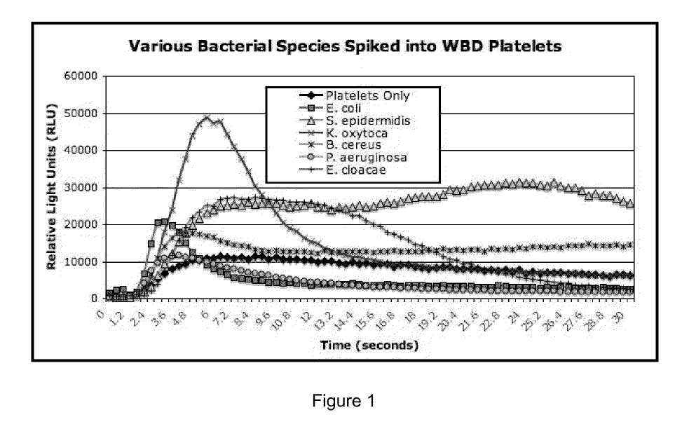

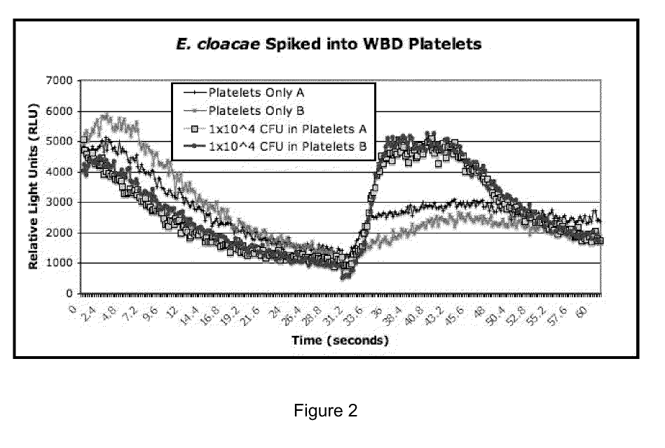

The final pellet is then mixed with a luciferin/luciferase reagent that does not contain any lytic reagents. The ATP production is monitored with a Berthold Sirius luminometer (Berthold Technologies GmbH& Co. KG, Bad Wildbad, Germany) for 30 seconds with readings taken every 0.2 seconds and total Relative Light Units (RLU) are recorded. After the first 30 seconds have elapsed, a second luciferin/luciferase reagent containing a bacterial lytic reagent is added, and the reaction is followed for another 30 seconds. The patterns of ATP production under these conditions permit the user to determine if bacteria are present in the sample. If bacteria are present, a specific signal is observed during the second 30-second reaction. If no bacteria are present, then the second 30-second reaction is similar to the first 30-second reaction. The amount of background ATP (RLUs) during the first reaction can be subtracted from or divided by the amount in the second reaction to estimate the number of bacteria present in the sample. Examples of this reaction scheme are shown in FIGS. 1 and 2.

In another particular embodiment, the present invention includes a method for the detection of bacteria in a beverage sample, e.g., beer or milk (final product or at any time during manufacture), where detection is accomplished using a two-step ATP luminescence reaction, essentially as described above, where the first step consists of determining background ATP levels without the use of a bacterial lysis reagent, and the second step consists of determining ATP levels while incorporating a bacterial lytic step that releases intracellular ATP. Bacterial ATP may be calculated by subtracting total extracellular ATP determined during the first step from total ATP determined during the second step. Bacterial ATP may be calculated by dividing an amount of ATP determined in the second step by an amount determined in the first step. The first step may include an ATP degrading enzyme such as apyrase to further reduce background ATP. In particular embodiments, the identity of bacteria can be identified by their unique ATP kinetic signal using time resolved light detection. The quantity of concentration of bacteria in the sample may also be determined by comparison to reference standards.

Typically, the invention contemplates the detection of a threshold level of bacteria in a sample. The skilled artisan would readily appreciate that the relevant threshold level depends, in large part, upon the sample being tested, the type of bacteria suspected of being present in the sample, and any industry or government-imposed standards related to maximum bacterial concentration. The determination of an appropriate threshold level for a particular sample to be tested may readily be determined by the skilled artisan based upon these and any other criteria established for a suitable application. Accordingly, the methods and devices of the invention may be optimized and/or the sensitivity adjusted such that a positive indication of the presence of bacteria in a sample occurs only when the amount of bacteria is above a certain threshold level. The sensitivity of the methods and devices of the invention may be adjusted by a variety of means well understood in the art, including, for example, by varying the concentration of one or more reagents used according to a method of the present invention, e.g., luciferin or luciferase. In certain embodiments, the threshold level is 1×10² organisms/ml, 5×10² organisms/ml, 1×10³ organisms/ml, 2×10³ organisms/ml, 5×10³ organisms/ml, 1×10⁴ organisms/ml, 2×10⁴ organisms/ml, 5×10⁴ organisms/ml, 1×10⁵ organisms/ml, or 5×10⁵ organisms/ml, or any integer value falling between. The threshold level may, alternatively be expressed as the number of colony forming units (cfu) present in a sample, and the threshold level may be, e.g., 50 cfu/ml, 1×10² cfu/ml, 5×10² cfu/ml, 1×10³ cfu/ml, 2×10³ cfu/ml, 5×10³ cfu/ml, 1×10⁴ cfu/ml, 2×10⁴ cfu/ml, 5×10⁴ cfu/ml, or 1×10⁵ cfu/ml, or any integer value falling between. Examples of suitable threshold levels for certain different samples include: milk: 5×10³ cfu/mL to 1×10⁵ cfu/mL; water: 10 cfu/mL to 1×10⁵ cfu/mL; blood: 10 cfu/mL to 1×10⁶ cfu/mL, urine: 10 cfu/mL to 1×10⁶ cfu/mL; fermenting corn mash: 5×10⁴ cfu/mL to 1×10⁸ cfu/mL; and beer: 10 cfu/mL to 1×10⁷ cfu/mL.

The present invention also provides reagents and kits that may be used to practice the methods of the present invention, e.g., to determine the presence of, amount of, or identity of bacteria present in a sample, e.g., a liquid sample. In particular embodiments, kits of the invention include reagents for use in a two-step ATP luminescence reaction of the present invention. This two-step assay comprises a first step of determining ATP activity in the absence of a lytic agent that lyses bacteria, and a second step of determining ATP activity in the presence of a lytic agent that lyses bacteria. Accordingly, in certain embodiments, a kit comprises reagents for performing an ATP luminescence assay in the presence of a lytic agent that lyses bacteria, as well as reagents for performing at an ATP luminescence assay in the absence of a lytic agent that lyses bacteria. In addition, in various embodiments, kits of the present invention may further comprise an agent that selectively lyses non-bacterial, e.g., eukaryotic or mammalian, cells. These reagents may be in separate vials or containers or may be combined in various manners suitable for storage etc. before use of the kit.

In particular embodiments, a kit of the present invention comprises a first container comprising luciferin and a second container comprising luciferase, wherein neither of these first or second containers includes a lytic agent that lyses bacteria. Either container may also comprise a divalent cation such as Mg²⁺ or Ca²⁺. The luciferin and luciferase are typically stored in separate containers and combined before use, in order to avoid any background conversion of the luciferin substrate. The contents of the first two containers (or portions thereof) may be combined for use in the first step of the two-step ATP luminescence assay, which does not include lysing bacteria. The kit further comprises a third container that comprises a lytic agent that lyses bacteria. The contents of the third container may be combined with portions of the contents of the first and second containers for use in the second step of the two-step ATP luminescence assay.

Alternatively, it may be convenient for the kit to comprise four containers, with the first two containers comprising luciferin and luciferase, respectively, wherein neither of these first or second containers includes a lytic agent that lyses bacteria, for combined use in the first step of the two-step ATP luminescence assay. The third and fourth container will also comprise luciferin and luciferase, respectively, but one of these containers will also comprise a lytic agent that lyses bacteria, for combined use in the second step of the two-step ATP luminescence assay. Either of the first or second and the third and fourth container may also comprise a divalent cation such as Mg²⁺ or Ca²⁺.

Embodiments of the kits of the present invention may also optionally include an additional container comprising a reagent that selectively lyses non-bacterial, e.g., eukaryotic or mammalian, cells. Embodiments of the kits of the present invention may also optionally include an additional container comprising an agent that reduces ATP activity, such as apyrase. In certain embodiments, kits comprise both an additional container comprising a reagent that selectively lyses non-bacterial, e.g., eukaryotic or mammalian, cells and an agent that reduces ATP activity.

Thus, in one particular embodiment, a kit of the present invention comprises:

1. a first container comprising luciferin, e.g., beetle luciferin;

2. a second container comprising luciferase, e.g., recombinant firefly luciferase, e.g., recombinant Photuris pennsylvanica luciferase; and

3. a third container comprising a bacterial lysis reagent, e.g., CHEX, CTAB, and/or an ethoxylated alkylphenol, such as Triton X-100, wherein said bacterial lysis reagent does not substantially interfere with the luciferin/luciferase reaction.

In another particular embodiment, a kit of the present invention comprises:

1. a first container comprising luciferin, e.g., beetle luciferin;

2. a second container comprising luciferase, e.g., recombinant firefly luciferase, e.g., recombinant Photuris pennsylvanica luciferase;

3. a third container comprising luciferin, e.g., beetle luciferin; and

4. a fourth container comprising luciferase, e.g., recombinant firefly luciferase, e.g., recombinant Photuris pennsylvanica luciferase,

wherein either the third or fourth container further comprises a bacterial lysis reagent, e.g., CHEX, CTAB, and/or an ethoxylated alkylphenol, such as Triton X-100, wherein said bacterial lysis reagent does not substantially interfere with the luciferin/luciferase reaction. In particular embodiments, both the third and fourth container further comprise the bacterial lysis reagent.

In one particular embodiment, a kit of the present invention comprises:

1. a first container comprising luciferin, e.g., beetle luciferin, and luciferase, e.g., recombinant firefly luciferase, e.g., recombinant Photuris pennsylvanica luciferase; and

2. a second container comprising a bacterial lysis reagent, e.g., CHEX, CTAB, and/or an ethoxylated alkylphenol, such as Triton X-100, wherein said bacterial lysis reagent does not substantially interfere with the luciferin/luciferase reaction.

In another particular embodiment, a kit of the present invention comprises:

1. a first container comprising luciferin, e.g., beetle luciferin, and luciferase, e.g., recombinant firefly luciferase, e.g., recombinant Photuris pennsylvanica luciferase; and

2. a second container comprising luciferin, e.g., beetle luciferin, and luciferase, e.g., recombinant firefly luciferase, e.g., recombinant Photuris pennsylvanica luciferase,

wherein either the first or second container further comprises a bacterial lysis reagent, e.g., CHEX, CTAB, and/or an ethoxylated alkylphenol, such as Triton X-100, wherein said bacterial lysis reagent does not substantially interfere with the luciferin/luciferase reaction. In particular embodiments, both the first and second container further comprise the bacterial lysis reagent.

In particular embodiments of the kits of the present invention, the bacterial lysis reagent is in a liquid. In particular embodiments of the kits of the present invention, the luciferin and/or the luciferase are freeze-dried. In various embodiments of the kits of the present invention, the luciferin and luciferase are present in different containers or in the same container.

In another particular embodiment, a kit of the present invention comprises:

1. a first container comprising luciferin, e.g., beetle luciferin;

2. a second container comprising luciferase, e.g., recombinant firefly luciferase, e.g., recombinant Photuris pennsylvanica luciferase;

3. a third container comprising a bacterial lysis reagent, e.g., CHEX, CTAB, and/or an ethoxylated alkylphenol, such as Triton X-100; and

4. a fourth container comprising an agent that selectively lyses non-bacterial, e.g., eukaryotic or mammalian, cells, e.g., Triton X-100.

In another particular embodiment, a kit of the present invention comprises:

1. a first container comprising luciferin, e.g., beetle luciferin;

2. a second container comprising luciferase, e.g., recombinant firefly luciferase, e.g., recombinant Photuris pennsylvanica luciferase;

3. a third container comprising luciferin, e.g., beetle luciferin; and

4. a fourth container comprising luciferase, e.g., recombinant firefly luciferase, e.g., recombinant Photuris pennsylvanica luciferase; and

5. a fifth container comprising an agent that selectively lyses non-bacterial, e.g., eukaryotic or mammalian, cells, e.g., Triton X-100, wherein either the third or fourth container further comprises a bacterial lysis reagent, e.g., CHEX, CTAB, and/or an ethoxylated alkylphenol, such as Triton X-100, wherein said bacterial lysis reagent does not substantially interfere with the luciferin/luciferase reaction. In particular embodiments, both the third and fourth container further comprise the bacterial lysis reagent.

In another particular embodiment, a kit of the present invention comprises:

1. a first container comprising luciferin, e.g., beetle luciferin;

2. a second container comprising luciferase, e.g., recombinant firefly luciferase, e.g., recombinant Photuris pennsylvanica luciferase;

3. a third container comprising a bacterial lysis reagent; and

4. a fourth container comprising an agent that selectively lyses non-bacterial, e.g., eukaryotic or mammalian, cells, e.g., Triton X-100; and

5. a fifth container comprising an agent that reduces ATP activity, e.g., apyrase.

In another particular embodiment, a kit of the present invention comprises:

1. a first container comprising luciferin, e.g., beetle luciferin;

2. a second container comprising luciferase, e.g., recombinant firefly luciferase, e.g., recombinant Photuris pennsylvanica luciferase;

3. a third container comprising luciferin, e.g., beetle luciferin;

4. a fourth container comprising luciferase, e.g., recombinant firefly luciferase, e.g., recombinant Photuris pennsylvanica luciferase;

5. a fifth container comprising an agent that selectively lyses non-bacterial, e.g., eukaryotic or mammalian, cells, e.g., Triton X-100; and

6. a sixth container comprising an agent that reduces ATP activity, e.g., apyrase,

wherein either the third or fourth container further comprises a bacterial lysis reagent, e.g., CHEX, CTAB, and/or an ethoxylated alkylphenol, such as Triton X-100, wherein said bacterial lysis reagent does not substantially interfere with the luciferin/luciferase reaction. In particular embodiments, both the third and fourth container further comprise the bacterial lysis reagent.

In another particular embodiment, a kit of the present invention comprises:

1. a first container comprising luciferin, e.g., beetle luciferin, and luciferase, e.g., recombinant firefly luciferase, e.g., recombinant Photuris pennsylvanica luciferase;

2. a second container comprising a bacterial lysis reagent, e.g., CHEX, CTAB, and/or an ethoxylated alkylphenol, such as Triton X-100; and

3. a third container comprising an agent that selectively lyses non-bacterial, e.g., eukaryotic or mammalian, cells, e.g., Triton X-100.

In another particular embodiment, a kit of the present invention comprises:

1. a first container comprising luciferin, e.g., beetle luciferin and luciferase, e.g., recombinant firefly luciferase, e.g., recombinant Photuris pennsylvanica luciferase;

2. a second container comprising luciferin, e.g., beetle luciferin, and luciferase, e.g., recombinant firefly luciferase, e.g., recombinant Photuris pennsylvanica luciferase; and

3. a third container comprising an agent that selectively lyses non-bacterial, e.g., eukaryotic or mammalian, cells, e.g., Triton X-100,

wherein either the first or second container further comprises a bacterial lysis reagent, e.g., CHEX, CTAB, and/or an ethoxylated alkylphenol, such as Triton X-100, wherein said bacterial lysis reagent does not substantially interfere with the luciferin/luciferase reaction. In particular embodiments, both the first and second container further comprise the bacterial lysis reagent.

In another particular embodiment, a kit of the present invention comprises:

1. a first container comprising luciferin, e.g., beetle luciferin, and luciferase, e.g., recombinant firefly luciferase, e.g., recombinant Photuris pennsylvanica luciferase;

2. a second container comprising a bacterial lysis reagent; and

3. a third container comprising an agent that selectively lyses non-bacterial, e.g., eukaryotic or mammalian, cells, e.g., Triton X-100; and

4. a fourth container comprising an agent that reduces ATP activity, e.g., apyrase.

In another particular embodiment, a kit of the present invention comprises:

1. a first container comprising luciferin, e.g., beetle luciferin, and luciferase, e.g., recombinant firefly luciferase, e.g., recombinant Photuris pennsylvanica luciferase;

2. a second container comprising luciferin, e.g., beetle luciferin, and luciferase, e.g., recombinant firefly luciferase, e.g., recombinant Photuris pennsylvanica luciferase;

3. a third container comprising an agent that selectively lyses non-bacterial, e.g., eukaryotic or mammalian, cells, e.g., Triton X-100; and

4. a fourth container comprising an agent that reduces ATP activity, e.g., apyrase,

wherein either the first or second container further comprises a bacterial lysis reagent, e.g., CHEX, CTAB, and/or an ethoxylated alkylphenol, such as Triton X-100, wherein said bacterial lysis reagent does not substantially interfere with the luciferin/luciferase reaction. In particular embodiments, both the first and second container further comprise the bacterial lysis reagent.

In one particular embodiment, the present invention provides a kit that may be used for detecting bacteria in a platelet preparation, comprising:

1. a first container comprising a solution of 0.16% Triton X-100 in distilled water;

2. a second container comprising a succinate buffer solution (e.g., 40 mM sodium succinate, 4 mM CaCl₂, 0.3% w/v BSA, pH 6.5);

3. a third container comprising apyrase (e.g., Sigma A6535) solution (e.g., 426 units/mL);

4. a fourth container comprising distilled sterile water;

5. a fifth container comprising a buffer solution without lytic reagents (e.g., 25 mM Hepes, 20 mM KCl, 2 mM EDTA, 0.01% w/v BSA, 5 mM MgCl₂, pH 7.75);

6. a sixth container comprising a buffer solution with a bacterial lysis agent, e.g., BacTiter-Glo™ Buffer with lytic reagent; and

7. a seventh container comprising freeze-dried luciferin/luciferase

(BacTiter-Glo™ Substrate).

In another embodiment, the above kit is provided without the container comprising distilled water. In another embodiment, the above kit is provided without the sixth and seventh container, which may be obtained separately as the BacTiter-Glo™ kit (Promega).

In various embodiments, the above kit may be provided with one or pieces of equipment useful in using the kit, such as, e.g., microcentrifuge tubes, a timer, a microcentrifuge, a Berthold Sirius Luminometer with auto-injector, a GenPrime microtube adapter, or analysis software.

In one particular embodiment, the present invention provides a kit that may be used to detect bacteria in corn mash, comprising:

1. a first container comprising a succinate buffer solution, e.g., 40 mM sodium succinate, 4 mM CaCl₂, 0.3% w/v BSA, pH 6.5;

2. a second container comprising apyrase (Sigma A6535) solution (e.g., 426 units/mL);

3. a third container comprising distilled sterile water;

4. a fourth container comprising a buffer solution without lytic reagents (e.g., 25 mM Hepes, 20 mM KCl, 2 mM EDTA, 0.01% w/v BSA, 5 mM MgCl₂, pH 7.75);

5. a fifth container comprising a buffer solution with bacterial lysis reagent, e.g., BacTiter-Glo™ Buffer with lytic reagents; and

6. a sixth container comprising freeze-dried luciferin/luciferase, e.g., BacTiter-Glo™ Substrate.

In another embodiment, the above kit is provided without the container comprising distilled water. In another embodiment, the above kit is provided without the sixth and seventh container, which may be obtained separately as the BacTiter-Glo™ kit (Promega).

In various embodiments, the above kit may be provided with one or pieces of equipment useful in using the kit, such as, e.g., a Swiss Gold coffee (SGC) filter, a 50 mL centrifuge tube, a 400 mL beaker, a stirring spoon, Microcon® (5 um pore size) centrifugal filter tubes, transfer pipettes, a timer, a microCentrifuge, 1.7 mL microcentrifuge tubes, a Berthold Sirius luminometer with auto-injector, a GenPrime microtube adapter, or analysis software.

In one particular embodiment, the present invention provides a kit that may be used to detect the presence of bacteria in beer, comprising:

1. a first container comprising a buffer solution without lytic reagents, e.g., 25 mM Hepes, 20 mM KCl, 2 mM EDTA, 0.01% w/v BSA, 5 mM MgCl₂, pH 7.75;

2. a second container comprising a buffer with a bacterial lysis reagent, e.g., BacTiter-Glo™ Buffer with lytic reagents; and

3. a third container comprising a freeze-dried luciferin/luciferase, e.g., BacTiter-Glo™ Substrate.

In various embodiments, the above kit may be provided with one or pieces of equipment useful in using the kit, such as, e.g., microcentrifuge tubes, a microCentrifuge, a timer, a Berthold Sirius luminometer with auto-injector, a GenPrime microtube adapter, or analysis software.

The kits of the present invention may optionally include additional components useful in practicing the present invention. For example, they may optionally include a tube for collecting, concentrating, or washing a liquid sample, e.g., a centrifuge tube, and/or a container for performing the ATP luminescence assays, e.g., a microfuge tube or a multiwall plate. In addition, they may optionally include a filter apparatus. They may also further optionally include other reagents, such as, e.g., sterile water or buffer for washing bacteria.

### Example 1

### Bacterial Detection in Whole Blood-Derived and Apheresis Platelet Concentrates Using ATP Luminescence

This example demonstrates that bacteria may be rapidly detected in platelet concentrates according to the methods of the present invention. Platelet concentrates were spiked with different amounts of various bacterial species as described below, and bacteria was detected in platelet concentrates using the following materials and protocol.

Materials:

1.7 mL Costar micro-centrifuge tubes

Lysis reagent, 0.16% v/v Triton X-100 (Reagent A)

Micro-centrifuge

Filter-sterile diH2O

Succinate Buffer, pH 6.5 with 0.3% BSA

Apyrase (Sigma A6535) at 495 Units/mL in diH₂O

Timer

Berthold Sirius luminometer

GenPrime microtube Adapter (GenPrime, Spokane, Wash., USA)

BacTiter-Glo™ Buffer Standard Reagent (Promega Corp., Madison, Wis., USA)

BacTiter-Glo™ Substrate (Promega Corp., Madison, Wis., USA)

BacTiter-Glo GenPrime Custom Reagent Buffer (BacTiter-Glo Standard Reagent without bacterial lysis agent) (GenPrime, Spokane, Wash., USA)

GenPrime BacSTAT Software (GenPrime, Spokane, Wash., USA)

Protocol:

Rehydrate one vial of BacTiter-Glo™ Substrate with 10 mL of GenPrime Custom Buffer to create Custom Reagent.

Rehydrate one vial of BacTiter-Glo™ Substrate with 10 mL of BacTiter-Glo™ Buffer to create BacTiter-Glo™ Standard Reagent.

Transfer 1 mL of platelet sample into 1.7 mL microtube.

Add 100 uL of Reagent A.

Invert for 1 minute.

Centrifuge tube for 3 minutes at 7500×g.

Gently pour off supernatant & blot tip of tube with towel.

Add 1 mL of diH2O and mix.

Centrifuge tube for 3 minutes at 7500×g.

Gently pour off supernatant & blot tip of tube with towel.

Add 200 ul of Succinate Buffer, pH 6.5 with 0.3% BSA.

Add 40 ul of Apyrase (Sigma A6535 @ 495 Units/mL in diH2O).

Incubate at room temperature for 10 minutes.

Add 1 mL of diH2O to tube and mix.

Centrifuge tube for 3 minutes at 7500×g.

Gently pour off supernatant & blot tip of tube with towel.

Resuspend in 200 uL of diH2O.

Add 50 ul of BacTiter-Glo Custom Reagent.

Insert tube into door of Luminometer and close door to start readings. Read every 0.2 seconds for 1 minute.

Inject 50 ul of BacTiter-Glo™ Standard reagent.

Read every 0.2 seconds for 30 seconds.

Analyze results using GenPrime software.

Experimental Design and Results

Three to five-day-old whole blood derived (WBD) platelets were collected and aliquoted into one-milliliter samples. These samples were then inoculated with known concentrations of bacteria, as determined by plate counts, or were left un-inoculated as negative controls. The bacteria selected for this experiment were known contaminants of blood products, e.g. P. aeruginosa and E. cloacae. The samples were then prepared following the previously mentioned experimental platelet protocol entitled: “Bacterial Detection in Whole Blood-Derived and Apheresis Platelet Concentrates Using ATP Luminescence”. The results were recorded as Relative Light Units (RLU) emitted by the reaction of the luciferin/luciferase reagent with ATP in the sample.

As shown in FIG. 1, different species of bacteria produced different kinetic curves of ATP bioluminescence over the 30 seconds following addition of the bacterial lysis agent, thus permitting the identification of contaminating bacterial species in platelet concentrates.

FIG. 2 demonstrates the pattern of ATP bioluminescence generated by platelet concentrate samples spiked with 1×10⁴ CFU of E. cloacae before and after introduction of the bacterial lysis agent at between 30 and 31 seconds. The results demonstrate the detection of E. cloacae ATP bioluminescence in the two spiked samples as compared to background ATP bioluminescence.

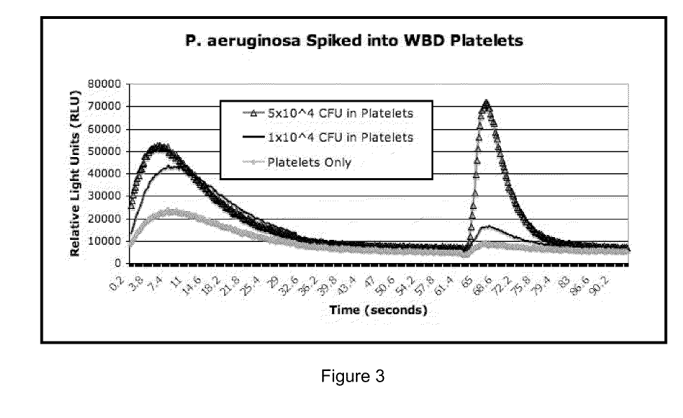

FIG. 3 similarly demonstrates the pattern of ATP bioluminescence generated by platelet concentrate samples spiked with either 1×10⁴ or 5×10⁴ CFU of P. aeruginosa before and after introduction of the bacterial lysis agent at 62 seconds. The results demonstrate the detection of P. aeruginosa ATP bioluminescence in the four spiked samples as compared to background ATP bioluminescence. In addition, this data demonstrates a relationship between the amount of bacteria present in the sample and the resulting bacterial ATP bioluminescence.

Overall, a significant correlation was observed between RLU and the concentration of bacteria in a platelet sample as determined by the control data. The sensitivity of bacterial detection was in the range of 5×10⁴ CFU/mL for all bacteria examined. No interfering substances were encountered for this assay.

Various parameters of this assay may be altered to increase the sensitivity of the assay, which include amplifying the ATP signal using a kinase catalyzed conversion of AMP+ADP to ATP.

The assay is simple to perform and takes less than 10 minutes per sample, as compared to the culture-based methods that can take up to 48 hours. Additionally, it requires minimal scientific skill and a small amount of equipment. This example demonstrates that the detection of ATP as an indicator of bacterial presence also allows for a robust assay that can detect any living microorganism.

### Example 2

### Bacterial Detection in Finished Beer Products Using ATP Luminescence

This example demonstrates that bacteria may be rapidly detected in finished beer products according to the methods of the present invention. Beer was spiked with different amounts of bacteria as described below, and bacteria were detected in the beer using the following materials and protocol.

Materials:

1.7 mL Costar Micro-centrifuge Tubes

Micro-centrifuge

Filter-sterile diH₂O

Timer

Berthold Sirius Luminometer

GenPrime Microtube Adapter (GenPrime, Spokane, Wash., USA)

BacTiter-Glo™ Buffer (Promega Corp., Madison, Wis., USA)

BacTiter-Glo™ Substrate (Promega Corp., Madison, Wis., USA)

GenPrime Custom Buffer (GenPrime, Spokane, Wash., USA)

GenPrime BacSTAT Software (GenPrime, Spokane, Wash., USA)

Protocol:

Rehydrate one vial of BacTiter-Glo™ Substrate with 10 mL of GenPrime Custom Buffer to create Custom Reagent.

Rehydrate one vial of BacTiter-Glo™ Substrate with 10 mL of BacTiter-Glo™ Buffer to create BacTiter-Glo™ Standard Reagent.

Transfer 1 mL of beer product sample into 1.7 mL microtube.

Centrifuge for 4 minutes at 7500×g.

Gently pour off supernatant.

Resuspend pellet in 100 uL of filter-sterile diH2O.

Add 50 ul of Custom Reagent

Insert tube into door of Luminometer and close door to start readings.

Read every 0.2 seconds for 1 minute.

Inject 50 uL of BacTiter-Glo™ Standard Reagent.

Read every 0.4 seconds for 5 minutes.

Analyze results using GenPrime Software.

Experimental Design and Results

One milliliter samples of finished beer product were collected and some samples were inoculated with either L. fermentum or L. acidophilus and were assayed for the presence of bacterial ATP. All samples were prepared according to the previously mentioned experimental protocol entitled: “Bacterial Detection in Finished Beer Products Using ATP Luminescence”. The amount of ATP was determined with a Berthold Sirius Luminometer (BERTHOLD TECHNOLOGIES GmbH& Co. KG Bad Wildbad/Germany) over a 300 second time period, with a bacterial lytic reagent being injected into the sample during this period. Results were recorded as relative light units (RLU) emitted from the reaction of the luciferin/luciferase reagents with ATP. Un-inoculated beer was used to determine background levels.

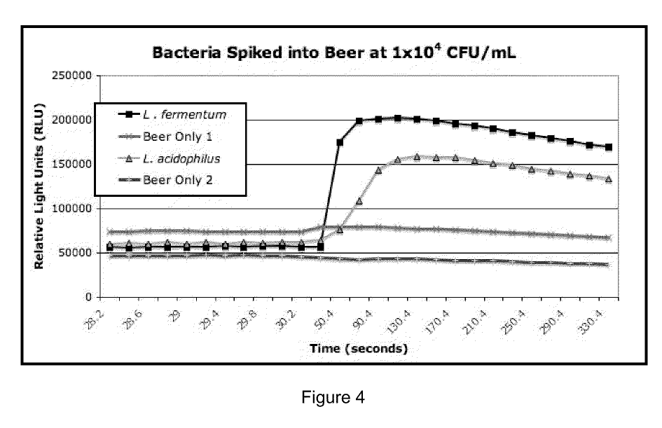

FIG. 4 demonstrates the kinetic curves of ATP bioluminescence of L. acidophilus and L. fermentum that have been inoculated into a finished beer product at a final concentration of 1×10⁴ CFU/mL after introduction of the bacterial lysis agent at approximately 31 seconds. In addition, this data demonstrates the detection of L. acidophilus and L. fermentum using ATP bioluminescence by comparing the kinetic curves of the test sample to the background level with un-inoculated beer.

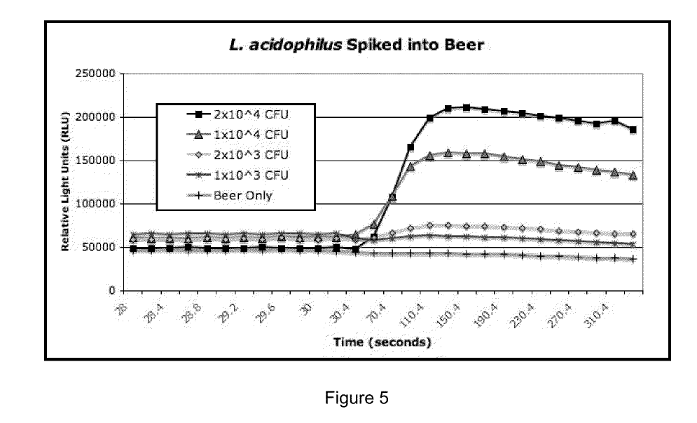

FIG. 5 demonstrates the kinetic curves of ATP bioluminescence of L. acidophilus spiked into a finished beer product at concentrations ranging from 1×10³ CFU/mL to 2×10⁴ CFU/mL after introduction of the bacterial lysis agent at approximately 31 seconds. This data demonstrates the sensitivity of detection of L. acidophilus in a finished beer product. In addition, this data demonstrates a direct relationship between the amount of bacteria present in the sample and the resulting bacterial ATP bioluminescence.

Overall, a significant correlation was observed between RLU and the concentration of bacteria in a beer sample. The sensitivity of bacterial detection was in the range of 2×10³ CFU/mL for both bacteria examined. No interfering substances were encountered for this assay.

### Example 3

### Bacterial Detection in Fermenting Corn Mash Using ATP Luminescence

This example demonstrates that bacteria may be rapidly detected in fermenting corn mash according to the methods of the present invention. Fermenting corn mash was spiked with different amounts of bacteria as described below, and bacteria were detected in the corn mash using the following materials and protocol.

Materials:

50 mL centrifuge tube

Swiss Gold coffee (SGC) filter

400 mL beaker

Stirring spoon

Microcon® (5 um pore size) Centrifugal Filter Tubes

Transfer Pipettes

Succinate Buffer, pH 6.5 with 0.3% BSA

Apyrase (Sigma A6535) at 495 Units/mL in diH2O

Timer

MicroCentrifuge

Berthold Sirius Luminometer

GenPrime Microtube Adapter (GenPrime, Spokane, Wash., USA)

BacTiter-Glo™ Buffer Standard Reagent (Promega Corp., Madison, Wis., USA)

BacTiter-Glo™ Substrate (Promega Corp., Madison, Wis., USA)

GenPrime Custom Buffer (GenPrime, Spokane, Wash., USA)

GenPrime BacSTAT Software (GenPrime, Spokane, Wash., USA)

Protocol:

Rehydrate one vial of BacTiter-Glo™ Substrate with 10 mL of GenPrime Custom Buffer to create Custom Reagent.

Rehydrate one vial of BacTiter-Glo™ Substrate with 10 mL of BacTiter-Glo™ Buffer to create BacTiter-Glo™ Standard Reagent.

Mix 20 mL of fresh mash in 50 mL centrifuge tube containing 20 mL of diH2O.

Place SGC filter into beaker and pour entire contents of 50 mL tube into filter.

Using a sterile spoon, stir mash until most of liquid has drained into beaker.

Pipette 200 ul of mash liquid and dispense into filter unit of Microcon® tube.

Close cap and place into centrifuge with cap hinge facing out, balance, and run for 8 minutes at 7500×g.

Discard filter and, using a transfer pipet, carefully remove supernatant.

Add 200 uL of Succinate Buffer, pH 6.5 with 0.3% BSA into tube.

Add 10 ul of apyrase (495 Units/mL).

Incubate for 10 minutes at room temperature.

Centrifuge tubes at 7500×g for 3 minutes.

Remove supernatant with pipet and resuspend in 100 ul of diH₂O.

Add 50 ul of Custom Reagent to tube

Insert tube into door of Luminometer and close door to start readings.

Read every 0.2 seconds for 1 minute.

Inject 50 ul of BacTiter-Glo™ Standard reagent.

Read every 4 seconds for 3 minutes.

Analyze result using GenPrime software.

Experimental Procedure and Results

Samples of yeast-fermenting corn mash inoculated with 5×10⁵ CFU/mL of Lactobacillus brevis or un-inoculated mash were assayed for the presence of bacterial ATP. All samples were prepared according to the previously mentioned experimental protocol entitled: “Bacterial Detection in Fermenting Corn Mash Using ATP Luminescence”. The amount of ATP bioluminescence was determined with a Berthold Sirius Luminometer ((BERTHOLD TECHNOLOGIES GmbH& Co. KG Bad Wildbad/Germany) during a 150 second time period. The bacterial lytic reagent was injected within that time period. Results were recorded as relative light units (RLU's) emitted from the reaction of the luciferin/luciferase reagents with ATP. Un-inoculated fermenting corn mash was used to determine background levels.

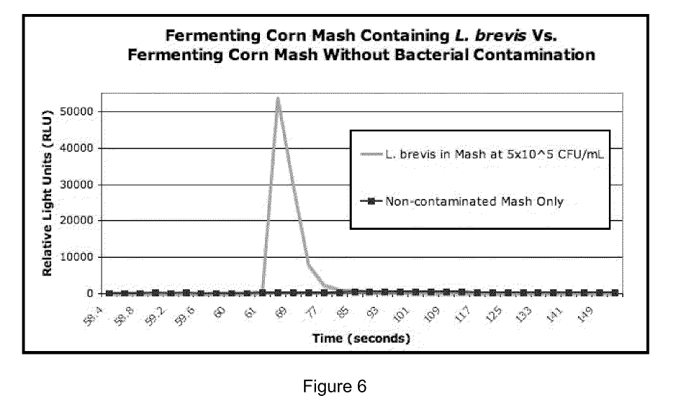

As shown in FIG. 6, bacterial ATP bioluminescence was clearly demonstrated in the spiked corn mash sample as compared to the background level associated with unspiked corn mash.

The sensitivity of bacterial detection was in the range of 1×10⁵ CFU/mL. No interfering substances were encountered for this assay.

### Example 4

### Bacterial Detection in Urine Using ATP Luminescence

This example provides an exemplary protocol for rapidly detecting the presence of bacteria in urine.

Materials:

1.7 mL Costar Micro-centrifuge Tubes

Cell Lysis reagent, 0.16% v/v Triton X-100 (Reagent A)

Micro-centrifuge

Filter-sterile diH2O

Succinate Buffer, pH 6.5 with 0.3% BSA

Apyrase (Sigma A6535) at 495 Units/mL in diH2O

Timer

Berthold Sirius Luminometer

GenPrime Microtube Adapter (GenPrime, Spokane, Wash., USA)

BacTiter-Glo™ Buffer (Promega Corp., Madison, Wis., USA)

BacTiter-Glo™ Substrate (Promega Corp., Madison, Wis., USA)

GenPrime Custom Buffer (GenPrime, Spokane, Wash., USA)

GenPrime BacSTAT Software (GenPrime, Spokane, Wash., USA)

Protocol:

Rehydrate one vial of BacTiter-Glo™ Substrate with 10 mL of GenPrime Custom Buffer to create Custom Reagent.

Rehydrate one vial of BacTiter-Glo™ Substrate with 10 mL of BacTiter-Glo™ Buffer to create BacTiter-Glo™ Standard Reagent.

Transfer 1 mL of urine sample into 1.7 mL microtube.

Centrifuge tube for 3 minutes at 7500×g.

Gently pour off supernatant & blot tip of tube with paper towel.

Add 200 ul of Succinate Buffer, pH 6.5 with 0.3% BSA.

Add 20 ul of Apyrase (Sigma A6535 @ 495 Units/mL in diH₂O).

Incubate at room temperature for 5 minutes.

Add 1 mL of diH₂O to tube and mix.

Centrifuge tube for 3 minutes at 7500×g.

Gently pour off supernatant & blot tip of tube with paper towel.

Resuspend in 200 uL of diH₂O.

Add 50 ul of Custom Reagent.

Insert tube into door of Luminometer and close door to start readings.

Read every 0.2 seconds for 1 minute.

Inject 50 ul of BacTiter-Glo™ Standard reagent.

Read every 0.2 seconds for 30 seconds.

Analyze result using GenPrime software.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1. Hoffman, R., Benz, E. J., Shattil, S. J. et al. Hematology: Basic Principles and Practice Edition 4, Elsevier Health Sciences, 2004.
2. Leytin, V., Allen, D. J., Hannach, B. and Freedman, Extension of human platelet storage up to 7-8 days does not reduce their in vivo viability in a rabbit model. J. Thrombosis and Haemostasis: supp. 1 Abstract No. P0070, 2003.
3. Sullivan, M. T. and Wallace, E. L. Blood collection and transfusion in the United States in 1999. Transfusion: 45: 141-148, 2005.
4. Benjamin, R. J. and Mintz, P. D. Bacterial detection and extended platelet storage: the next step forward. Transfusion: 45:1832-1835, 2005.
5. Ribault, S. Harper, K., Grave, L., Lafontaine, C., Nannini, P., Raimondo, A., and Besson Faure, I. Rapid screening method for detection of bacteria in platelet concentrates. J. Clinical Microbiology, 42: 1903-1908, 2004.
6. Yomtovian, R. A., Palavecino, E. L., Dykstra, A. H., Downes, K. A., Morrissey, A. M., Bajaksouzian, S., Pokorny, M. A., Lazarus, H. M., and Jacobs, M. R. Evolution of surveillance methods for detection of bacterial contamination of platelets in a university hospital, 1991-2004. Transfusion: 46:719-730, 2006.
7. Hillyer, C. D., Josephson, C. D. Blajchman, M. A., Vostal, J. G., Epstein, J. S. and Goodman, J. L. Bacterial contamination of blood components: risks, strategies, and regulation, joint ASH and AABB educational session in transfusion medicine. Hematology: 575-589, 2003.
8. Nilsson, L. E., Molin, O., Ansehn, S. Bioluminescent assay of bacterial ATP for rapid detection of bacterial growth in clinical blood cultures. J. Bioluminescence and Chemiluminescence Vol 3: 101-104, 1989.
9. Strehler, Bernard L. “Bioluminescence Assay: Principles and Practice”. Methods of Biochemical Analysis. Ed. David Glick. Vol. 16. Wiley, 1968.
10. Ericson, D. G. Rapid and sensitive detection of bacteria in blood products, urine and other fluids. U.S. Pat. No. 7,419,798, 2008.

1. A method for detecting the presence of or determining an amount of bacteria in a sample, comprising:
(a) determining a first amount of ATP activity present in a sample;
(b) contacting the sample of step (a) with a lytic agent at a concentration and for a time sufficient to lyse bacteria in said sample; and
(c) determining a second amount of ATP activity present in the sample concurrent with or following step (b),
thereby determining the presence of bacteria in the sample.

2. (canceled)

3. The method of claim 1, further comprising reducing the first amount of ATP activity.

4. The method of claim 3, wherein the first amount of ATP activity is reduced before step (a).

5. The method of claim 3, wherein the first amount of ATP activity is reduced after step (a) and before step (b).

6. The method of claim 1, further comprising contacting the sample with a lytic agent at a concentration and for a time sufficient to lyse mammalian cells but not bacteria before step (a).

7. A method for determining the presence of or an amount of bacteria in a sample, comprising:
(a) reducing ATP activity present in a sample;
(b) contacting the sample with a lytic agent at a concentration and for a time sufficient to lyse bacteria in said sample after step (a); and
(c) determining an amount of ATP activity present in the sample concurrent with or following step (b),
thereby determining the presence of bacteria in the sample.

8. (canceled)

9. A method for identifying the type of bacteria in a sample, comprising:
(a) reducing ATP activity present in a sample;
(b) contacting the sample of step (a) with a lytic agent at a concentration and for a time sufficient to lyse bacteria in said sample following step (a); and
(c) determining the rate of ATP activity present in the sample concurrent with or following step (b),
thereby identifying the type of bacteria in the sample.

10. A method for detecting the presence of bacteria in a sample, comprising contacting a sample with a lytic agent at a concentration and for a time sufficient to lyse bacteria in said sample and concurrently determining an amount of ATP activity present in the sample.

11. The method of claim 1, wherein the sample is a liquid sample.

12. The method of claim 11, wherein the liquid sample is a biological sample.

13. The method of claim 12, wherein the biological sample is selected from the group consisting of: blood, plasma, urine, platelets, bronchial lavage, saliva, wound fluid, tears, cerebrospinal fluid, amniotic fluid, and pleural fluid.

14. The method of claim 11, wherein the liquid sample is a food or beverage.

15. The method of claim 14, wherein the food or beverage is a dairy product.

16. The method of claim 15, wherein the beverage is an alcoholic beverage.

17. The method of claim 16, wherein the alcoholic beverage is beer.

18. The method of claim 11, wherein the liquid sample is fermenting corn mash.

19. The method of claim 11, wherein the liquid sample is a cell culture.

20. The method of claim 1, wherein the amounts of ATP activity are determined using a luciferin/luciferase reagent.

21. The method of claim 1, wherein step (b) and step (c) are performed concurrently.

22. The method of claim 21, wherein step (b) and step (c) are performed concurrently using a luciferin/luciferase reagent containing a lytic reagent.

23. The method of claim 1, wherein the presence or amount of bacteria is determined using time-resolved light detection to determine an ATP kinetic profile.

24. The method of claim 1, wherein the presence of bacteria is determined where the second amount of ATP activity is significantly greater than the first amount of ATP activity.

25. The method of claim 1, wherein the amount of bacteria is determined by a method comprising subtracting the first amount of ATP activity from the second amount of ATP activity.

26. The method of claim 1, wherein the amount of bacteria is determined by a method comprising dividing the second amount of ATP activity by the first amount of ATP activity.

27. The method of claim 1, wherein the bacteria is selected from the group consisting of: Staphylococcus epidermis, Staphylococcus aureus, Bacillus cereus, Bacillus subtilis, Pseudomonas aeruginosa, Steptococcus viridans/mitis, Streptococcus pyogenes, Escherichia coli, Klebsiella oxytoca, Serratia marcescens, Enterobacter cloacae, Clostridium perfringens, Corynebacterium xerosis, Propionibacterium acnes, Salmonella spp., Lactobacillus rhamnosus, Lactobacillus fermentum, Lactobacillus paracasei, Lactobacillus brevis, Lactobacillus acidophilus, Lactobacillus plantarum, Lactobacillus crispatus, Lactobacillus delbrueckii, Pediococcus acidilactici, Pediococcus pentosaceus, Weisella confusa, Leuconostoc citreum, Leuconostoc mesenteroides, Leuconostoc lactis, Acetobacter spp., Gluconobacter oxydans, Serratia liquefaciens, klebsiella pheumoniae, Providencia stuartii, Providencia rettgeri, Providencia alcalifaciens, Proteus mirabilis, Proteus vulgaris, Morganella morganii, and Enterococcus faecalis.

28. The method of claim 1, further comprising providing the sample to be tested for the presence of bacteria before step (a).

29. The method of claim 28, wherein the sample is a liquid sample.

30. A kit for detecting the presence of a microorganism in a sample, comprising:
(a) a first container comprising luciferin and luciferase;
(b) a second container comprising a bacterial lysis reagent; and
(c) a third container comprising an agent that selectively lyses a non-bacterial cell.

31. A kit for detecting the presence of a microorganism in a sample, comprising:
(a) a first container comprising a buffer solution without a bacterial lysis reagent;
(b) a second container comprising a buffer solution with a bacterial lysis reagent; and
(c) a third container comprising luciferin and luciferase.
